(19) United States
(12) Patent Application Publication
(10) Pub. No.: US 2023/0077910 A1
(43) Pub. Date: Mar. 16, 2023

(54) ELECTROOSMOTIC PUMP

(71) Applicant: EOFLOW CO., LTD., Seongnam-si (KR)

(72) Inventors: Jae Hong KIM, Namyangju-si (KR); Young Wook CHANG, Seoul (KR); Mihyun LEE, Seongnam-si (KR)

(73) Assignee: EOFLOW CO., LTD., Seongnam-si (KR)

(21) Appl. No.: 17/988,159

(22) Filed: Nov. 16, 2022

Related U.S. Application Data
(63) Continuation-in-part of application No. 17/652,139, filed on Feb. 23, 2022.
Application No. 17/652,139 is a continuation of application No. 16/326,768, filed on Feb. 20, 2019, now Pat. No. 11,286,918.

(30) Foreign Application Priority Data
Aug. 31, 2016 (KR) 10-2016-0112131
Jul. 18, 2022 (KR) 10-2022-0088383

(51) Int. Cl. F04B 19/00; A61M 39/24; A61M 5/142; A61M 5/145

(52) U.S. Cl. CPC F04B 19/006; A61M 39/24; F04B 19/00; A61M 5/142; A61M 5/145; B01L 2400/0418

(57) ABSTRACT

An electro-osmosis pump system includes an inlet line through which a fluid is introduced, an outlet line through which the fluid is discharged, a first pump disposed between the inlet line and the outlet line and including a first housing in which a first operation fluid is disposed, a second pump disposed in parallel to the first pump between the inlet line and the outlet line and including a second housing in which a second operation fluid disposed, and a power supply configured to supply voltages to the first pump and the second pump. The first pump includes a first membrane, a 1A-th electrode, and a 2A-th electrode, the second pump includes a second membrane, a 1B-th electrode, and a 2B-th electrode, and the power supply supplies the voltage to the 1A-th electrode and the 2A-th electrode, and supplies the voltage to the 1B-th electrode and the 2B-th electrode.

### CROSS-REFERENCE TO RELATED APPLICATIONS

This application claims priority to Korean Patent Application No. 10-2022-0088383 filed on Jul. 18, 2022 and is a continuation-in-part of U.S. patent application Ser. No. 17/652,139 filed on Feb. 23, 2022, which is a continuation of U.S. patent application Ser. No. 16/326,768, filed on Feb. 20, 2019 and issued as U.S. Pat. No. 11,286,918, as the U.S. National Phase under 35 U.S.C. § 371 of International Application PCT/KR2017/008292, filed Aug. 1, 2017, which claims priority to Korean Patent Application No. 10-2016-0112131, filed Aug. 31, 2016. Each of these applications is hereby incorporated by reference in its entirety.

### TECHNICAL FIELD

The disclosure relates to a patch-type liquid medicine delivery device attached to a human body, or an electro-osmosis pump and an electro-osmosis pump system for transmission of liquid medicine, which may be applied to a wearable medical device. The disclosure also relates to a dialysis system with which blood is dialyzed.

### BACKGROUND ART

An electro-osmosis pump is a pump that uses a phenomenon of fluid movement which occurs when a voltage is applied to opposite ends of a capillary or a porous separation membrane. The electro-osmosis pump can be manufactured to be very small in size compared to a general mechanical pump, and generates no noise and consumes less power.

For example, international patent publication No. WO 2011/112723 discloses an electro osmosis pump that includes a ceramic membrane disposed between a porous sliver/oxidized silver anode and a porous sliver/oxidized cathode.

The above-stated patent discloses a liquid medicine delivery device in which electrodes are formed at opposite ends of the ceramic separation membrane, and when a predetermined potential is applied to the electrodes, a pressure that corresponds to the corresponding potential is generated and the device uses the pressure. The liquid medicine delivery device additionally uses two electric fluid chambers and a needle for injection of liquid medicine.

However, the electro osmosis pump used in the liquid medicine delivery device has a structure in which liquid medicine to be delivered and water, which is an operation fluid used for driving of the pump are separated from each other by oil, and thus unexpected mixing of the liquid medicine and water may occur at any time in an interface between the liquid medicine and water.

In the electro osmosis pump, liquid medicine, which is an object to be delivered, and water, which is an operation fluid, need to be separated from each other. Liquid medicine is a mixture of various substances, and a biologically active substance in components of the mixture may be oxidized or reduced in a potential for driving of the electro osmosis pump. In addition, when the liquid medicine directly contacts the electrode, a high molecular substance such as protein is adsorbed to the electrodes, thereby deteriorating performance of the pump, and accordingly, it is preferable that the electrodes and the liquid medicine need to be separated from each other.

However, a conventional method cannot sufficiently satisfy such a condition.

In addition, an amount of power to be consumed for driving of the pump is a factor that determines practical utility when the pump is applied to a patch-type liquid medicine delivery device or a wearable medical device attached to a human body, and accordingly, a small-sized pump, which can be driven with low power, is required.

### SUMMARY

### Technical Problem

The disclosure has been made in an effort to resolve the above-described problems, and provides an electro osmosis pump that can be stably driven by preventing liquid medicine, which is an object to be delivered, and operation fluid from being mixed with each other, thereby improving merchantability, and can prevent a side effect thereby the therapeutic effect for a patient.

In addition, another purpose of the disclosure is to provide an electro osmosis pump that can maximize fluid transfer efficiency with respect to power consumption. The disclosure provides a liquid medicine injection device which is safely driven and can accurately transfer a liquid medicine.

Another objective of the disclosure is to provide an electro-osmosis pump system and a dialysis system, capable of continuously providing a pumped fluid by alternately pumping the fluid at opposite ends.

Another objective of the disclosure is to provide an electro-osmosis pump system and a dialysis system, capable of increasing a flow rate pumped by pumps disposed in parallel.

### Technical Solution

In order to achieve such a purpose, the disclosure provides an electro-osmosis pump system including an inlet line through which a fluid is introduced, an outlet line through which the fluid is discharged, a pump disposed between the inlet line and the outlet line and including a first housing in which a first operation fluid is disposed, a second pump disposed in parallel to the first pump between the inlet line and the outlet line and including a second housing in which a second operation fluid disposed, and a power supply configured to supply voltages to the first pump and the second pump, wherein the first pump includes a first membrane disposed in the first housing, a 1A-th electrode disposed on one side of the first membrane, and a 2A-th electrode disposed on the other side of the first membrane, the second pump includes a second membrane disposed in the second housing, a 1B-th electrode disposed on one side of the second membrane, and a 2B-th electrode disposed on the other side of the second membrane, and the power supply supplies the voltage to the 1A-th electrode and the 2A-th electrode of the first pump by alternating polarities, and supplies the voltage to the 1B-th electrode and the 2B-th electrode of the second pump by alternating polarities, wherein the polarity applied to the 1A-th electrode of the first pump and the polarity applied to the 1B-th electrode of the second pump are different from each other.

Further, the first operation fluid of the first pump and the second operation fluid of the second pump may move in different directions.

Further, the first pump may further include a 1A-th diaphragm disposed to be spaced apart from the 1A-th electrode, a 1A-th check valve configured to move the fluid from the inlet line to the 1A-th diaphragm, and a 2A-th check valve configured to move the fluid from the 1A-th diaphragm to the outlet line, and the second pump may further include a 1B-th diaphragm disposed to be spaced apart from the 1B-th electrode, a 1B-th check valve configured to move the fluid from the inlet line to the 1B-th diaphragm, and a 2B-th check valve configured to move the fluid from the 1B-th diaphragm to the outlet line.

Further, a discharge cycle of the fluid discharged from the 2A-th check valve of the first pump and a discharge cycle of the fluid discharged from the 2B-th check valve of the second pump may be different from each other.

Further, in the first pump and the second pump, the fluid may be alternately introduced from the inlet line and alternately discharged to the outlet line.

Further, the first pump may discharge the fluid from the inlet line to the outlet line in a first cycle, and the second pump may discharge the fluid from the inlet line to the outlet line in a second cycle different from the first cycle.

Further, the electro-osmosis pump system may further include a third pump disposed between the inlet line and the outlet line in parallel to the first pump and in parallel to the second pump, wherein the power supply may supply a voltage to the third pump by alternating polarities, wherein the polarity may be applied in a cycle that is different from a cycle of the polarity applied to the first pump and a cycle of the polarity applied to the second pump.

Further, a flow rate of the fluid discharged from the third pump may be different from a flow rate of the fluid discharged from the first pump or the second pump.

Another aspect of the disclosure provides a dialysis system including a first line through which one of blood and dialysate moves, a second line through which the other one of the blood and the dialysate moves, a dialysis device through which the first line and the second line pass and in which the blood is dialyzed by the dialysate, a pump module disposed in at least one of the first line and the second line and configured to move at least one of the dialysis and the blood passing therethrough, and a power supply configured to alternately supply power to the pump module by alternating polarities.

Further, the pump module may include a first pump disposed in the first line and including a first housing in which an operation fluid disposed, and a second pump disposed in the first line in parallel to the first pump and including a second housing in which an operation fluid disposed, wherein the first pump may include a first membrane disposed in the first housing, a 1A-th electrode disposed on one side of the first membrane, and a 2A-th electrode disposed on the other side of the first membrane, the second pump may include a second membrane disposed in the second housing, a 1B-th electrode disposed on one side of the second membrane, and a 2B-th electrode disposed on the other side of the second membrane, and the power supply may supply a voltage to the 1A-th electrode and the 2A-th electrode of the first pump by alternating polarities, and supply a voltage to the 1B-th electrode and the 2B-th electrode of the second pump by alternating polarities, wherein the polarities may be applied to the 1A-th electrode of the first pump and the 1B-th electrode of the second pump with different cycles.

Further, the operation fluid of the first pump and the operation fluid of the second pump may move in different directions.

Further, the first pump may further include a 1A-th diaphragm disposed to be spaced apart from the 1A-th electrode, a 1A-th check valve configured to move the blood or the dialysate from the first line to the 1A-th diaphragm, and a 2A-th check valve configured to move the blood or the dialysate from the 1A-th diaphragm to the first line, the second pump may further include a 1B-th diaphragm disposed to be spaced apart from the 1B-th electrode, a 1B-th check valve configured to move the blood or the dialysate from the first line to the 1B-th diaphragm, and a 2B-th check valve configured to move the blood or the dialysate from the 1B-th diaphragm to the first line, and a discharge cycle of the blood or the dialysate discharged from the 2A-th check valve of the first pump or a discharge cycle of the blood or the dialysate discharged from the 2B-th check valve of the second pump may be different from each other.

Further, the first pump may discharge the blood or the dialysate into the first line in a first cycle, and the second pump may discharge the blood or the dialysate in a second cycle different from the first cycle.

Further, the dialysis system may further include a third pump disposed in parallel to the first pump and in parallel to the second pump, wherein the power supply may supply a voltage to the third pump by alternating polarities, wherein the polarity may be applied in a cycle that is different from a cycle of the polarity applied to the first pump and a cycle of the polarity applied to the second pump.

Further, a flow rate of the blood or the dialysate discharged from the third pump may be different from a flow rate of the blood or the dialysate discharged from the first pump or the second pump.

Further, the pump module may include at least one pump,

wherein the pump may include a housing, a driver including a first membrane disposed in the housing, a first electrode disposed on one side of the first membrane, and a second electrode disposed on the other side of the first membrane, a diaphragm assembly including a first diaphragm disposed on one side of the driver and a second diaphragm disposed on the other side of the driver, a first valve assembly mounted to face the first diaphragm and including a first check valve disposed at an inlet end and a second check valve disposed at an outlet end, and a second valve assembly mounted to face the second diaphragm and including a third check valve disposed at an inlet end and a fourth check valve disposed at an outlet end.

Further, the pump may be disposed in the first line, and one of the blood and the dialysate may be alternately introduced into the first check valve and the third check valve and alternately discharged from the second check valve and the fourth check valve.

Further, in the pump, the first valve assembly may be disposed in the first line, the second valve assembly may be disposed in the second line, one of the blood and the dialysate may be discharged from the second check valve, and the other one of the blood and the dialysate discharged from the fourth check valve are alternately discharged.

### Advantageous Effects

According to the present invention, a pump operation fluid and a fluid to be delivered such as liquid medicine are separated from each other by a flexible diaphragm and thus an active component included in the fluid to be delivered can be prevented from being spoiled due to an electro-chemical reaction by a voltage applied to an electrode.

In addition, according to the present invention, a component included in the pump operation fluid can be prevented from being fixed with the liquid medicine, and thus various transfer fluids can be applied in design of a patch-type liquid medicine delivery device, thereby increasing the degree of freedom in a design. That is, the disclosure enables the design of a patch-type liquid medicine delivery device that can selectively supply an optimum liquid medicine to a patient suffering from a specific disease such as diabetes, or can be applied to patients suffering from various diseases.

Further, the disclosure provides an effect of improving merchantability of the electro osmosis pump driven with low power and high efficiency by applying a check valve having a very low opening pressure and increasing a reaction speed of the check valve.

According to the present invention, valve assemblies can be disposed on opposite sides of a pump, and can alternately pump a fluid by alternately receiving polarities. Thus, the pump can continuously provide the pumped fluid, so that a flow rate can be increased. A dialysis system to which the pump is applied can increase dialysis efficiency by consecutively proving a driving force to blood or dialysate.

According to the present invention, pumps are disposed in parallel, so that a pumped flow rate can be increased. Since a flow rate discharged from each of the pumps is alternately joined to each other, a large flow rate can be continuously provided.

### DESCRIPTION OF THE DRAWINGS

FIG. 1 is a perspective view of an external appearance of an electro osmosis pump provided for description of an exemplary embodiment of the present invention.

FIG. 2 is a perspective view of the electro osmosis pump of FIG. 1, viewed from a different angle.

FIG. 3 is an exploded perspective view of FIG. 1.

FIG. 4 is a cross-sectional view of FIG. 1, taken along the line IV-IV.

FIG. 5 is a cross-sectional view of FIG. 1, taken along the line V-V.

FIG. 6 is a view illustrating a check valve assembly of an exemplary embodiment mountable to the pump of FIG. 1.

FIG. 7 is a perspective view illustrating a modified example of the check valve assembly of FIG. 6.

FIG. 8 is a cross-sectional view illustrating a pump according to an exemplary embodiment of the present invention.

FIG. 9 is a view illustrating a dialysis system according to an exemplary embodiment of the present invention.

FIG. 10 is a view illustrating a pump module of an exemplary embodiment, which is applied to FIG. 9.

FIG. 11 is a graph illustrating a flow rate of the pump module of FIG. 10.

FIG. 12 is a view illustrating a pump module of another exemplary embodiment, which is applied to FIG. 9.

FIG. 13 is a graph illustrating a flow rate of the pump module of FIG. 12.

FIG. 14 is a graph illustrating a flow rate of a pump module of another exemplary embodiment, which is applied to FIG. 9.

FIG. 15 is a view illustrating a pump module of another exemplary embodiment, which is applied to FIG. 9.

FIG. 16 is a view illustrating a dialysis system according to another exemplary embodiment of the present invention.

FIG. 17 is a view illustrating a pump module of an exemplary embodiment, which is applied to FIG. 16.

FIG. 18 is a view illustrating a pump module of another exemplary embodiment, which is applied to FIG. 16.

### DETAILED DESCRIPTION

The disclosure may include various exemplary embodiments and modifications, and certain exemplary embodiments thereof are illustrated in the drawings and will be described herein in detail. The effects and features of the disclosure and the accomplishing methods thereof will become apparent from the following description of the exemplary embodiments taken in conjunction with the accompanying drawings. However, the disclosure is not limited to the exemplary embodiments described below, and may be implemented in various modes.

Hereinafter, the exemplary embodiments of the disclosure will be described in detail with reference to the accompanying drawings. In the following description, like reference numerals will denote like configuration elements, and redundant descriptions thereof will be omitted.

In the following descriptions of the exemplary embodiments, the terms of a singular form may include plural forms unless referred to the contrary.

It will be further understood that the terms “comprises” and/or “comprising” used herein specify the presence of stated features or configuration elements, but do not preclude the presence or addition of one or more other features or configuration elements.

The order of processes explained in one embodiment may be changed in a modification of the exemplary embodiment or another exemplary embodiment. For example, two consecutively described processes may be performed substantially at the same time or performed in an order opposite to the described order.

In the drawings, the sizes of configuration elements may be exaggerated for clarity. In other words, since sizes and thicknesses of elements in the drawings are arbitrarily illustrated for convenience of explanation, the following exemplary embodiments are not limited thereto.

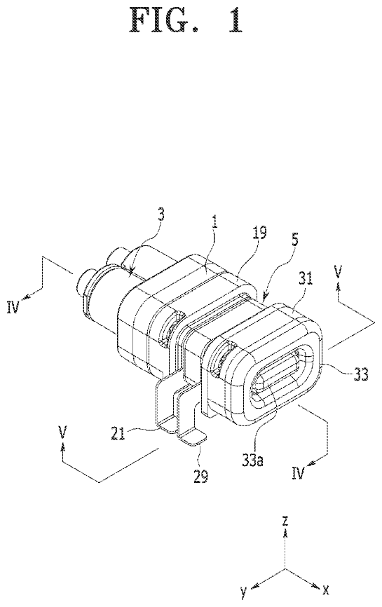

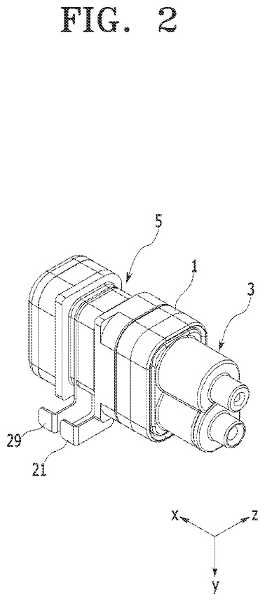

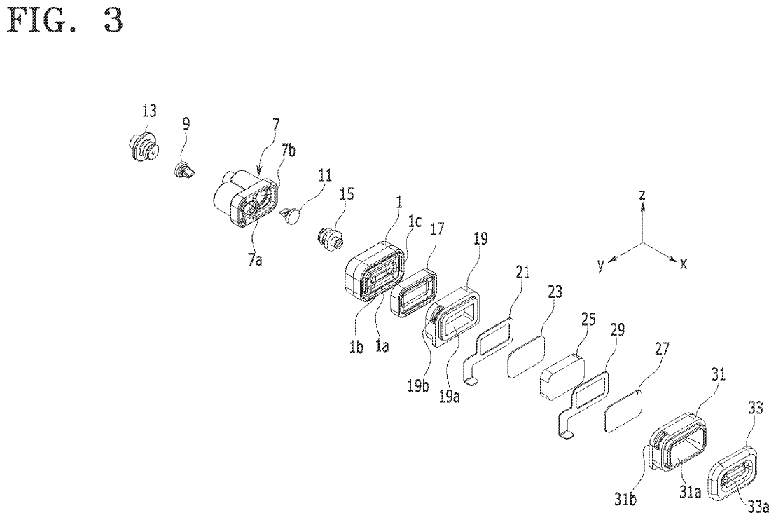

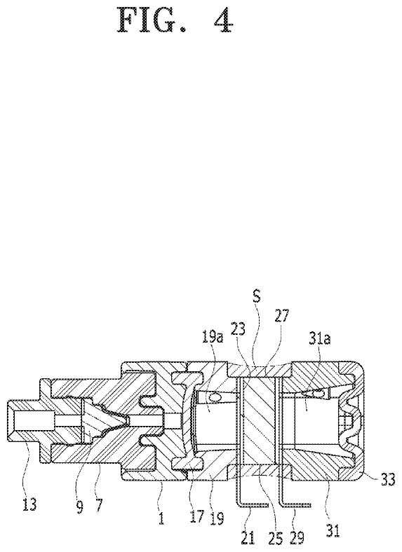

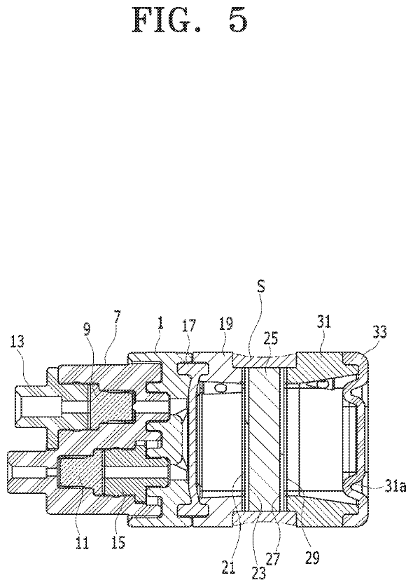

FIG. 1 and FIG. 2 are perspective view provided for description of an exemplary embodiment of the present invention, FIG. 3 is an exploded perspective view of FIG. 1, FIG. 4 is a cross-sectional view of FIG. 1, taken along the line IV-IV, and FIG. 5 is a cross-sectional view of FIG. 1, taken along the line V-V, which illustrates an electro-osmosis pump.

An electro-osmosis pump according to an exemplary embodiment of the disclosure includes a connector 1, a check valve assembly 3, and a driver 5.

The check valve assembly 3 may be combined to one side of the connector 1 and the driver may be combined to the other side of the connector 1. The connector 1 is provided with a barrier rib 1a that partitions the check valve assembly 3 and the driver 5. The barrier rib 1a includes a liquid medicine inlet 1b and a liquid medicine outlet 1c.

The liquid medicine inlet 1b and the liquid medicine outlet 1c are disposed at a distance from each other, and penetrate the barrier rib 1a.

Liquid medicine to be injected into a human body is put into the liquid medicine inlet 1b, and the liquid medicine outlet 1c may be a path through which the liquid medicine put into the liquid medicine inlet 1b is discharged so as to be injected back into the human body.

The check valve assembly 3 may include a valve housing 7, an inflow check valve 9, a discharge check valve 11, a first fixing hole 13, and a second fixing hole 15.

The valve housing 7 includes a liquid medicine inflow extension pipeline 7a and a liquid medicine discharge extension pipeline 7b. The valve housing 7 may be combined to one side of the connector 1. In addition, the liquid medicine inflow extension pipeline 7a is connected to the liquid medicine inlet 1b, and the liquid medicine discharge extension pipeline 7b is connected to the liquid medicine outlet 1c.

The inflow check valve 9 is disposed in the liquid medicine inflow extension pipeline 7a. The inflow check valve 9 may let liquid medicine injected into a human body pass to the liquid medicine inlet 1b and block movement of the liquid medicine in the reverse direction.

The discharge check valve 11 is disposed in the liquid medicine discharge extension pipeline 7b. The discharge check valve 11 may let the liquid medicine passed through the liquid medicine inlet 1b pass in a direction of injection into the human body, and blocks movement in the reverse direction.

As the inflow check valve 9 and the discharge check valve 11, duckbill valves that are flexible and have a low open-pressure may be used. Fluid transmission efficiency compared to a power consumption amount is increased by the inflow check valve 9 and the discharge check valve 11, thereby enabling long-time operation and improving productivity.

The first fixing hole 13 is fitted into the liquid medicine inflow extension pipeline 7a to fix the inflow check valve 9. The second fixing hole 15 is fitted into the liquid medicine discharge extension pipeline 7b to fix the discharge check valve 11.

In addition, the first fixing hole 13 and the second fixing hole 15 preferably have pipelines through which liquid medicine can pass.

In the exemplary embodiment of the present invention, the inflow check valve 9 and the discharge check valve 11 are combined to the valve housing 7, but this is not restrictive, and the inflow check valve 9 and the discharge check valve 11 may be respectively combined to the liquid medicine inlet 1b and the liquid medicine outlet 1c provided in the connector 1. In this case, the valve housing 7 is integrally formed with the connector 1 so that it can be manufactured with a simpler structure. According to the other exemplary embodiment of the present invention, the number of parts is reduced so that manufacturing cost can be more saved, and the product can be manufactured to be more compact.

The driver 5 is combined to one side of the connector 1. The driver 5 is preferably disposed opposite to a side where the check valve assembly 3 is combined. The driver 5 is preferably disposed apart from the liquid medicine that passes through the check valve assembly 3. The driver 5 applies pressure to the liquid medicine passed through the check valve assembly 3 such that the liquid medicine can pass through the liquid medicine outlet 1c.

The driver 5 may include a first diaphragm 17, a first pump housing 19, a first power supply line 21, a first electrode 23, a membrane 25, a second electrode 27, a second power supply line 29, a second pump housing 31, and a second diaphragm 33.

The first diaphragm 17 is combined to one side of the connector 1. A space is provided between the first diaphragm 17 and the connector 1. That is, the first diaphragm 17 is combined to the connector 1, while maintaining a certain space in the connector 1. Thus, liquid medicine at the check valve assembly 3 maintains a state of being isolated rather than moving toward the driver 5 by the first diaphragm 17.

The first diaphragm 17 of which a plane that forms the first diaphragm 17 can iteratively move in a predetermined section in an axial direction by a pressure generated from the driver 5. A wrinkle portion may be provided in the first diaphragm 17 to allow the plane to smoothly move along the axial direction (i.e., the x-axis direction in FIG. 1).

The first diaphragm 17 is combined to one side of the first pump housing 19. The first pump housing 19 is provided with a space 19a that penetrates along an axial direction. Thus, one side of the space 19a of the first pump housing 19 may be closed by the first diaphragm 17.

The first electrode 23 is combined to the other side of the first pump housing 19 so that the space 19a formed by the first pump housing 19 may be closed. In addition, the first pump housing 19 may accommodate operation fluid such as water and the like in the space 19a provided therein.

The first pump housing 19 may be provided with a fluid injection hole portion 19b at an external circumference thereof. Such a hole portion 19b may be sealed after the operation fluid is injected into the first pump housing 19. Thus, the operation fluid of the driver 5 may be separated from the liquid medicine at the check valve assembly 3.

The first power supply line 21 may supply power to the first electrode 23. The first power supply line 21 is disposed along an edge of the first pump housing 19, and may be fixed to the first electrode 23 by contacting the same. The first power supply line 21 is preferably disposed between the first pump housing 19 and the first electrode 23. However, according to another exemplary embodiment of the present invention, the first power supply line 21 may supply power to the first electrode 23, and may be disposed between the first electrode 23 and the membrane 25.

The first electrode 23 is formed in the shape of a plate and thus may close the space 19a of the first pump housing 19. That is, the first pump housing 19 may form the space 19a with the first diaphragm 17 and the first electrode 23. In addition, the operation fluid such as water and the like is accommodated in the space 19a of the first pump housing 19.

The membrane 25 may be formed of a porous material through which the operation fluid and ions can be transferred. The membrane 25 is preferably made of an insulator such as a ceramic and the like. When the membrane 25 is formed of an insulator, an electro-chemical reaction material used in the first electrode 23 and the second electrode 27 is consumed or desorbed due to long-term driving of the electro-osmosis pump and thus the porous membrane 25 is exposed. However, in this case, even when conventional carbon paper or carbon cloth is used, a side reaction such as electrolysis of water, which occurs due to exposure to carbon paper or carbon cloth, does not occur. Thus, unnecessary power consumption due to a side reaction can be prevented. Therefore, according to the present invention, a safe driving characteristic can be provided and durability can be improved.

The membrane 25 may be used by processing a flexible non-conductive material such as a polymer resin, rubber, urethane, or a plastic film into a thin film form.

The second electrode 27 is disposed on the other side of the membrane 25. That is, the membrane 25 is preferably disposed between the first electrode 23 and the second electrode 27. The second power supply line 29 may supply external power to the second electrode 27. The second power supply line 29 may be combined to an edge of the second pump housing 31. However, the second power supply line 29 may have any alignment structure as long as it has a structure for supplying power to the second electrode 27.

The shape of the second pump housing 31 is the same as or similar to the shape of the first pump housing 19. Another space 31a that penetrates the second pump housing 31 along an axial direction is provided in the second pump housing 31. As in the first pump housing 19, a hole portion 31b that penetrates the space 31a may be provided in the second pump housing 31. The hole portion 31b of the second pump housing 31 may be sealed by a sealant or filled by welding and the like after operation fluid is injected therein.

The second diaphragm 33 is combined to one side of the second pump housing 31 and thus may close the space 31a provided in the second pump housing 31.

That is, the second pump housing 31 may close the space 31a by using the second electrode 27, which is formed in the shape of a plate, and the second diaphragm 33.

A wrinkle portion 33a may be formed in a plane of the second diaphragm 33. The wrinkle portion 33a formed in the second diaphragm 33 may be formed of protrusions and depressions that protrude in the axial direction with reference to a cross-section. The wrinkle portion 33a of the second diaphragm 33 enhances performance of pumping by sufficiently moving the plane of the second diaphragm 33 along the axial direction.

In the exemplary embodiment of the present invention, the wrinkle portion 33a is formed in the second diaphragm 33, but depending on exemplary embodiments, a wrinkle portion may be also formed in the first diaphragm 17. In addition, a wrinkle portion that can be formed in the first diaphragm 17 or the second diaphragm 33 maximizes deformation of the first diaphragm 17 and the second diaphragm 33 with small energy, thereby reducing energy consumption. That is, the driver 5 can be driven for a long period of time with a small external power source.

As shown in FIG. 4 and FIG. 5, the above-described first pump housing 19, the first power supply line 21, the first electrode 23, the membrane 25, the second electrode 27, the second power supply line 29, and the second pump housing 31 may be air-tightly sealed from the outside by an encapsulant S. That is, the first power supply line 21, the first electrode 23, the membrane 25, the second electrode 27, and the second power supply line 29 are formed relatively smaller than the first pump housing 19 and the second pump housing 31 in size, and thus the encapsulant S may be disposed in a circumferential portion (i.e., a portion exposed to the outside and a portion that forms a groove or a space with reference to a cross-section) between the first pump housing 19 and the second pump housing 31 while the first power supply line 21, the first electrode 23, the membrane 25, the second electrode 27, and the second power supply line 29 are in an assembled state. Such an encapsulant S may form an encapsulation layer that maintains air-tight encapsulation from the outside.

As the encapsulant, an adhesive such as a hot melt adhesive, an epoxy adhesive, a polyurethane adhesive, or a cyanoacrylate adhesive may be used. However, the encapsulant is not limited to such examples, and any material that is rigidly cured to prevent leakage of operation fluid and prevent deformation of an external appearance of a configuration element is applicable.

An operation process of the above-described exemplary embodiment of the disclosure will now be described in detail.

First, power is supplied such that the first power supply line 21 and the second power supply line 29 have different polarities, and a voltage difference occurs between the first electrode 23 and the second electrode 27. Due to such a voltage difference, positive ions are generated as a result of an electrode reaction in an anode. The positive ions generated from the above-stated reaction move to a cathode and pass through the membrane 25 while pulling the operation fluid together such that a pressure (a pumping force) is generated.

That is, such an electrochemical reaction enables ions and the operation fluid to move to the space 19a of the first pump housing 19 or the space 31a of the second pump housing 31 by passing through the membrane 25.

When the polarity of power of the first electrode 23 and the polarity of power of the second electrode 27 are alternately supplied through the first power supply line 21 and the second power supply line 29, the operation fluid can be iteratively moved to the space 19a of the first pump housing 19 and the space 31a of the second pump housing 31 by the above-described electrochemical reaction.

That is, when an electrode which functions as an anode is changed to serve as a cathode due to alternation of the voltage polarity, an electro-chemical reactant consumed when the electrode is used as an anode can be recovered when the electrode is used as a cathode, and vice versa. Accordingly, the electro-osmosis pump can be continuously driven.

Then, the first diaphragm 17 and the second diaphragm 33 are deformed and a pressure is generated. Such a pressure is applied to a space between the connector 1 and the first diaphragm 17.

Then, the liquid medicine is introduced into the liquid medicine inlet 1b through the liquid medicine inflow extension pipeline 7a by the pressure. The introduced liquid medicine may be injected into a human body while being discharged along the liquid medicine outlet 1c and the liquid medicine discharge extension pipeline 7b.

In this case, the inflow check valve 9 and the discharge check valve 11 allow the liquid medicine to move along only one direction. Thus, the electro-osmosis pump according to the exemplary embodiment of the disclosure can safely inject liquid medicine into a human body while using low power.

In particular, since the operation fluid of the driver 5 and the liquid medicine are separated from each other, the electro-osmosis pump of the exemplary embodiment of the disclosure can prevent an active component included in the liquid medicine from being spoiled due to an electro-chemical reaction.

In addition, according to the present invention, a component included in the operation fluid of the driver 55 can be prevented from being transmitted to the liquid medicine such that a wider range of liquid medicine or operation fluid is applicable.

In addition, according to the present invention, a check valve of which an opening pressure is very low is used so that a reaction speed of the check valve is very fast, and accordingly, the pump can be driven with low power and high efficiency as a whole.

The electro-osmosis pump of the disclosure is applicable to an electro-osmosis pump system or a dialysis system to be described below.

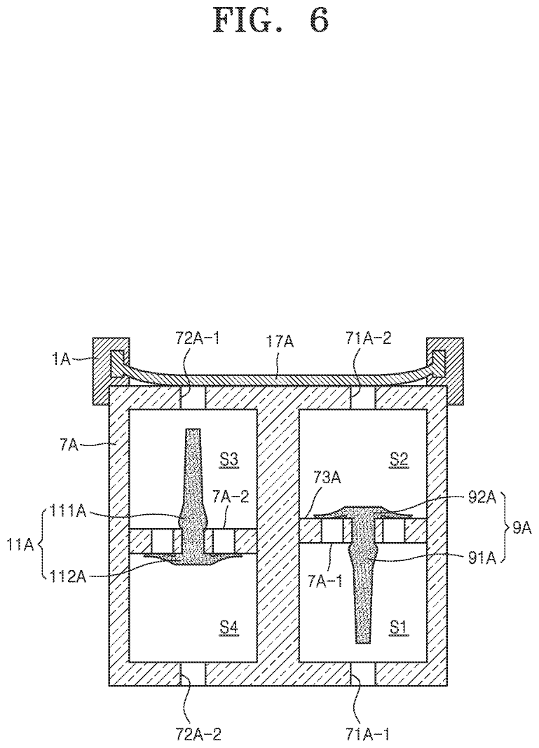

FIG. 6 is a view illustrating a check valve assembly of an exemplary embodiment mountable to the pump of FIG. 1.

Referring to FIG. 6, the check valve assembly is mountable to the electro-osmosis pump of the above-described exemplary embodiment or a valve assembly of a pump 100 to be described below. The check valve assembly may include a valve housing 7A, a first check valve 9A, and a second check valve 11A.

The check valve assembly may be mounted to face a first diaphragm 17A, and may be mounted to a housing, in which a driver (not shown) is installed, through a connector 1A.

The first check valve 9A and the second check valve 11A that allows a fluid to move in different directions may be disposed in the valve housing 7A.

The first check valve 9A may be disposed between a first space S1 and a second space S2 of the valve housing 7A. The first check valve 9A may allow a fluid to pass through the first space S1 and the second space S2 from the outside and move to the first diaphragm 17A. The fluid may pass through a first-first opening 71A-1 to move from the outside to the first space S1, and pass through a first-second opening 71A-2 to move from the second space S2 to the first diaphragm 17A.

The first check valve 9A may include a first shaft 91A and a first head 92A. The first shaft 91A may be disposed in the first space S1, and the first head 92A may be disposed in the second space S2. The first shaft 91A may be inserted into a first barrier rib 73A that divides the first space S1 and the second space S2, and the first head 92A may selectively open and close a first through hole 7A-1 disposed in the first barrier rib 73A.

The second check valve 11A may be disposed between a third space S3 and a fourth space S4 of the valve housing 7A. The second check valve 11A may allow the fluid to pass through the third space S3 and the fourth space S4 from the first diaphragm 17A and move to the outside. The fluid may pass through a second-first opening 72A-1 to move from the first diaphragm 17A to the third space S3, and pass through a second-second opening 72A-2 to move from the fourth space S4 to the outside.

The second check valve 11A may include a second shaft 111A and a second head 112A. The second shaft 111A may be disposed in the third space S3, and the second head 112A may be disposed in the fourth space S4. The second shaft 111A may be inserted into a second barrier rib 74A that divides the third space S3 and the fourth space S4, and the second head 112A may selectively open and close a second through hole 7A-2 disposed in the second barrier rib 74A.

When a pressure of a space between the first diaphragm 17A and the valve housing 7A is lowered, the fluid is introduced into the first space S1 through the first-first opening 71A-1. The first head 92A opens the first through hole 7A-1 since a pressure of the first space S1 is higher than that of the second space S2, and the second head 112A closes the second through hole 7A-2 since a pressure of the third space S3 is lower than that of the fourth space S4. The fluid passes through the first through hole 7A-1 to move to the second space S2, and then passes through the first-second opening 71A-2 to move to the space between the first diaphragm 17A and the valve housing 7A.

When the pressure of the space between the first diaphragm 17A and the valve housing 7A increases, the fluid is discharged to the outside through the second-second opening 72A-2. The first head 92A closes the first through hole 7A-1 since the pressure of the first space S1 is lower than that of the second space S2, and the second head 112A opens the second through hole 7A-2 since the pressure of the third space S3 is higher than that of the fourth space S4. The fluid moves from the first diaphragm 17A to the third space S3 through the second-first opening 72A-1, passes through the second through hole 7A-2 to move to the fourth space S4, and then passes through the second-second opening 72A-2 to be discharged to the outside.

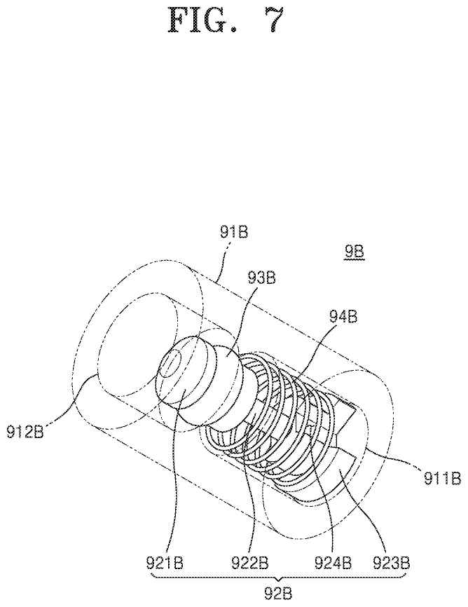

FIG. 7 is a perspective view illustrating a modified example of the check valve assembly of FIG. 6.

Referring to FIG. 7, a check valve assembly 9B may include a tube 91B, a body 92B, a sealing 93B, and an elastic member 94B. The check valve assembly 9B may be installed in the first check valve 9A and the second check valve 11A of FIG. 6.

The body 92B may be inserted into the tube 91B, and the tube 91B may be divided into a first space 911B and a second space 912B. The first space 911B and the second space 912B may be divided by a neck formed by narrowing an inner diameter of the tube 91B. A shaft 922B of the body 92B may be disposed in the first space 911B, and a head 921B of the body 92B may be disposed in the second space 912B. In the check valve assembly 9B, a fluid may move from the first space 911B of the tube 91B to the second space 912B thereof.

The body 92B may include the head 921B, the shaft 922B, a flange 923B, and a groove 924B. The shaft 922B, the flange 923B, and the groove 924B may be disposed in the first space 911B, and the head 921B may be disposed in the second space 912B.

The head 921B may be inserted into the second space 912B, and the sealing 93B may be provided on the head 921B. Due to the sealing 93B, a flow from the second space 912B to the first space 911B may be blocked.

The shaft 922B may extend from the head 921B and may be inserted into the first space 911B. The flange 923B may be disposed on an end portion of the head 921B.

The flange 923B may receive the pressure of the fluid to move the shaft 922B. In addition, when the flange 923B is inserted through the elastic member 94B and receives the pressure of the fluid, the elastic member 94B may be compressed.

When the fluid in the first space 911B has a higher pressure than the fluid in the second space 912B, the fluid in the first space 911B may press a rear end of the flange 923B. In this case, the groove 924B may be disposed on one side of the flange 923B so that the fluid can pass through the groove 924B and move to the shaft 922B.

Since the check valve assembly 9B includes the flange 923B, which receives the pressure of the fluid so that the body 92B moves, and the groove 924B, which allows the fluid to move therethrough, the opening and closing of the check valve assembly 9B can be simply operated according to the pressure of the fluid, and the fluid can move when the check valve assembly 9B is opened.

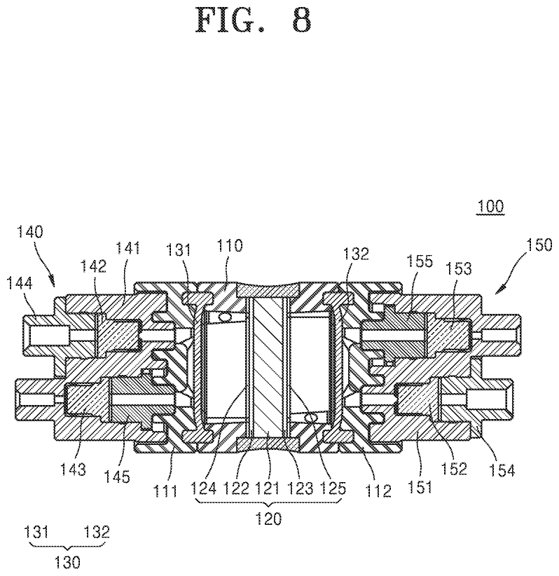

FIG. 8 is a cross-sectional view illustrating a pump according to an exemplary embodiment of the present invention.

Referring to FIG. 8, the pump 100 may be driven by an electro-osmotic method. The pump 100 may include diaphragms disposed on both sides thereof and thus may pump fluid alternately and continuously.

The pump 100 may include a housing 110, a driver 120, a diaphragm unit 130, a first valve assembly 140, and a second valve assembly 150.

An operation fluid may be stored in the housing 110 The driver 120 may be disposed in the housing 110, and the diaphragm unit 130 may be disposed on an end portion of the housing 110.

The driver 120 may include a membrane 121, a first electrode 122, a second electrode 123, a first electrode tab 124, and a second electrode tab 125.

The membrane 121 may be made of a porous material through which an operation fluid and ions move. The membrane 121 may be substantially the same as the membrane 25 described above.

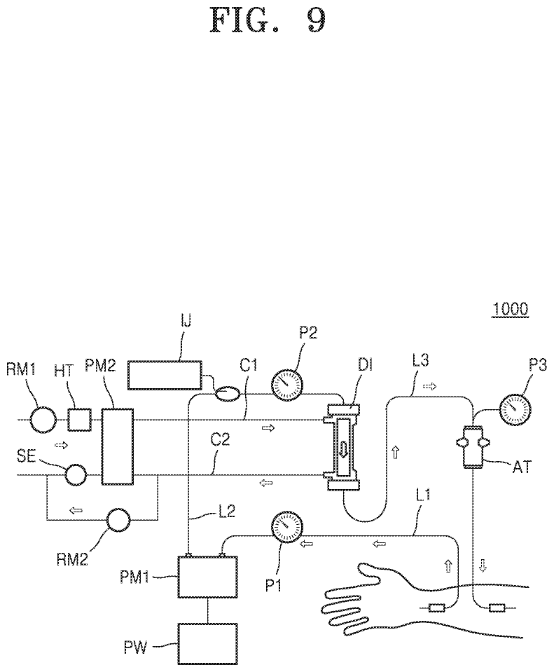

The first electrode 122 may be disposed on one side of the membrane 121, and the second electrode 123 may be disposed on the other side of the membrane 121. The first electrode 122 may be electrically connected to a power supply PW of FIG. 9 due to the first electrode tab 124. The second electrode 123 may be electrically connected to the power supply PW due the second electrode tab 125.

The diaphragm unit 130 may include a first diaphragm 131 and a second diaphragm 132. By the diaphragm unit 130, the operation fluid may be allowed to move in the housing 110.

The first diaphragm 131 may cover one side of the housing 110 and may be disposed adjacent to the first valve assembly 140. One end of the housing 110 may have a first space V1 covered by the first diaphragm 131 and the first electrode 122.

The second diaphragm 132 may cover the other side of the housing 110 and may be disposed adjacent to the second valve assembly 150. The other end of the housing 110 may have a second space V2 covered by the second diaphragm 132 and the second electrode 123.

The first valve assembly 140 may be mounted on one end of the housing 110 and may be disposed to face the first diaphragm 131. The first valve assembly 140 may be mounted to the housing 110 by a first connector 111.

In an exemplary embodiment, the first valve assembly 140 may have a first valve housing 141, a first check valve 142, and a second check valve 143. The first valve housing 141, the first check valve 142, and the second check valve 143 may be the valve housing 7, the inflow check valve 9, and the discharge check valve 11, respectively, described above.

In an alternative exemplary embodiment, the first valve assembly 140 may include a first fixing hole 144 and a second fixing hole 145. The first fixing hole 144 and the second fixing hole 145 may be the first fixing hole 13 and the second fixing hole 15, respectively, described above.

The second valve assembly 150 may be mounted on the other end of the housing 110 and may be disposed to face the second diaphragm 132. The second valve assembly 150 may be mounted to the housing 110 by a second connector 112.

In an exemplary embodiment, the second valve assembly 150 may include a second valve housing 151, a third check valve 152, and a fourth check valve 153. The second valve housing 151, the third check valve 152, and the fourth check valve 153 may be the valve housing 7, the inflow check valve 9, and the discharge check valve 11, respectively, described above.

In an alternative exemplary embodiment, the second valve assembly 150 may include a third fixing hole 154 and a fourth fixing hole 155. The third fixing hole 154 and the fourth fixing hole 155 may be the first fixing hole 13 and the second fixing hole 15, respectively, described above.

The power supply PW may alternately supply voltages of different polarities to the first electrode 122 and the second electrode 123. When the voltages of different polarities are alternately supplied to the first electrode 122 and the second electrode 123, a voltage difference is generated between the first electrode 122 and the second electrode 123. Due to such a voltage difference, positive ions are generated in an anode by an electrode reaction, and the generated positive ions move to a cathode and pass through the membrane 121 while pulling the operation fluid together, thereby generating a pressure.

Since the polarities are alternately applied to the first electrode 122 and the second electrode 123, flow directions of the operation fluid may be alternately changed from the first space V1 to the second space V2 and from the second space V2 to the first space V1.

When the operation fluid moves from the first space V1 to the second space V2, the first diaphragm 131 moves toward the driver 120, the first check valve 142 is opened, and the second check valve 143 is closed. At the same time, the second diaphragm 132 moves away from the driver 120, the third check valve 152 is closed, and the fourth check valve 153 is opened. That is, an inlet of the first valve assembly 140 is opened and an outlet thereof is closed, but an inlet of the second valve assembly 150 is closed and an outlet thereof is opened.

When the operation fluid moves from the second space V2 to the first space V1, the first diaphragm 131 moves away from the driver 120, the first check valve 142 is closed, and the second check valve 143 is opened. At the same time, the second diaphragm 132 moves toward the driver 120, the third check valve 152 is opened, and the fourth check valve 153 is closed. That is, the inlet of the first valve assembly 140 is closed and the outlet thereof is opened, but the inlet of the second valve assembly 150 is opened and the outlet thereof is closed.

In the pump 100, the first valve assembly 140 and the second valve assembly 150 may alternately introduce fluid and alternately discharge the pumped fluid. A cycle in which the fluid is introduced and discharged by the first valve assembly 140 and a cycle in which the fluid is introduced and discharged by the second valve assembly 150 are set to be different from each other.

In detail, since the polarities are alternately applied to the first electrode 122 and the second electrode 123 of the driver 120, the cycle in which the fluid is introduced and discharged by the first valve assembly 140 may be opposite to the cycle in which the fluid is introduced and discharged by the second valve assembly 150.

In an exemplary embodiment, the same fluid may be introduced into and discharged from the first valve assembly 140 and the second valve assembly 150, and thus the pump 100 can continuously pump the fluid. In the pump 100, the fluid may be alternately introduced into the first valve assembly 140 and the second valve assembly 150, and then the fluid may be alternately discharged from the first valve assembly 140 and the second valve assembly 150, and thus the pump 100 can continuously pump the fluid within a preset variation range.

In an exemplary embodiment, different fluids may be introduced into and discharged from the first valve assembly 140 and the second valve assembly 150, respectively, so that the pump 100 can pump heterogeneous fluids. In the pump 100, a first fluid may be introduced into the first valve assembly 140 and discharged from the first valve assembly 140, and a second fluid may be introduced into the second valve assembly 150 and discharged from the second valve assembly 150. In this case, the first valve assembly 140 and the second valve assembly 150 can simultaneously pump heterogeneous fluids by alternately pumping the heterogeneous fluids.

Since the diaphragms are disposed on opposite sides of the driver 120, respectively, and the first valve assembly 140 and the second valve assembly 150 are driven in conjunction with each other, the efficiency of the pump 100 can be increased.

FIG. 9 is a view illustrating a dialysis system according to an exemplary embodiment of the present invention.

Referring to FIG. 9, a dialysis system 1000 may include a first line through which blood moves and a second line through which dialysate moves. The first line and the second line pass through a dialysis device DI, and the blood may be dialyzed from the dialysis device DI.

The dialysis system 1000 may include a pump system for pumping blood or dialysate. In an exemplary embodiment, the pump system may be an electro-osmosis pump system. A pump module in the following description may be replaced with the pump system or the electro-osmosis pump system.

A first pump module PM1 may be disposed in the first line and move blood. A second pump module PM2 may be disposed in the second line and move dialysate. The above-described electro-osmosis pump of FIG. 1 or pump 100 of FIG. 8 may be disposed in the first pump module PM1 and the second pump module PM2.

The first line may include a 1A-th line L1 extending from a patient to the first pump module PM1, a 1B-th line L2 extending from the first pump module PM1 to the dialysis device DI, and a 1C-th line L3 extending from the dialysis device DI to the patient.

In an alternative exemplary embodiment, a first pressure gauge P1 may be disposed in the 1A-th line L1 and may measure a pressure of the blood flowing into the first pump module PM1. A second pressure gauge P2 may be disposed in the 1B-th line and may measure a pressure of the blood pumped by the first pump module PM1. A third pressure gauge P3 may be disposed in the 1C-th line L3 and may measure a pressure of the blood passing through the dialysis device DI and moving back to the patient.

In an alternative exemplary embodiment, a liquid medicine injector IJ may be disposed in the first line. The liquid medicine injector IJ may be disposed in the 1B-th line L2, and an anticoagulant such as heparin may be injected into the liquid medicine injector IJ. For example, the liquid medicine injector IJ may be disposed between the first pump module PM1 and the second pressure gauge P2.

In an alternative exemplary embodiment, an air trap AT may be disposed in the first line. The air trap AT may remove air from the dialyzed blood, or may detect air excessively included in the blood. The air trap AT may be disposed in the 1C-th line L3.

The second line may include a 2A-th line C1, which extends from a first reservoir (not shown) to the dialysis device DI and through which the dialysate before dialysis moves, and a 2B-th line C2 which extends from the dialysis device DI to a second reservoir (not shown) and through which the dialysate after the dialysis moves.

The second pump module PM2 may be disposed such that the 2A-th line C1 and the 2B-th line C2 pass therethrough. Due to such an arrangement, the second pump module PM2 may pump the dialysate moving to the dialysis device DI and the dialysate discharged from the dialysis device DI, thereby transferring driving force with which the dialysate can move.

In an exemplary embodiment, as the second pump module PM2, the pump 100 described above may be applied. When the 2A-th line C1 is disposed in the first valve assembly 140 and the 2B-th line C2 is disposed in the second valve assembly 150, dialysate may receive driving force due to the driving of the pump 100 and move in each of the 2A-th line C1 and the 2B-th line C2. The second pump module PM2 may transmit driving force to both the dialysate introduced into the dialysis device DI and the dialysate discharged from the dialysis device DI so that a circulation rate of the dialysate is increased, thereby increasing dialysis efficiency of the dialysis system 1000.

In an alternative exemplary embodiment, the second line may include an air removal pump RM1. The air removal pump RM1 may be disposed in the 2A-th line C1. The air removal pump RM1 may remove air included in the dialysate before flowing into the dialysis device DI.

In an alternative exemplary embodiment, the second line may include a heater HT. The heater HT may be disposed in the 2A-th line C1. The heater HT may set a temperature of dialysate before flowing into the dialysis device DI to a predetermined range. For example, the heater HT may set the temperature of the dialysate to be substantially equal to a temperature of the blood. Since the dialysate is maintained at a preset temperature by the heater HT, a change in temperature of the blood passing through the dialysis device DI can be minimized.

In an alternative exemplary embodiment, the second line may include a moisture removal pump RM2. The moisture removal pump RM2 may be disposed in the 2B-th line C2. The moisture removal pump RM2 may remove moisture of the dialysate discharged from the dialysis device DI. In an exemplary embodiment, the moisture removal pump RM2 may be disposed in a branch line branched from the 2B-th line C2. One end of the branch line may be connected between the second pump module PM2 and the dialysis device DI, and the other end of the branch line may be connected to a discharge end of the second pump module PM2.

In an alternative exemplary embodiment, the second line may include a detector SE. The detector SE may be disposed in the 2B-th line C2 and may detect blood included in the dialysate.

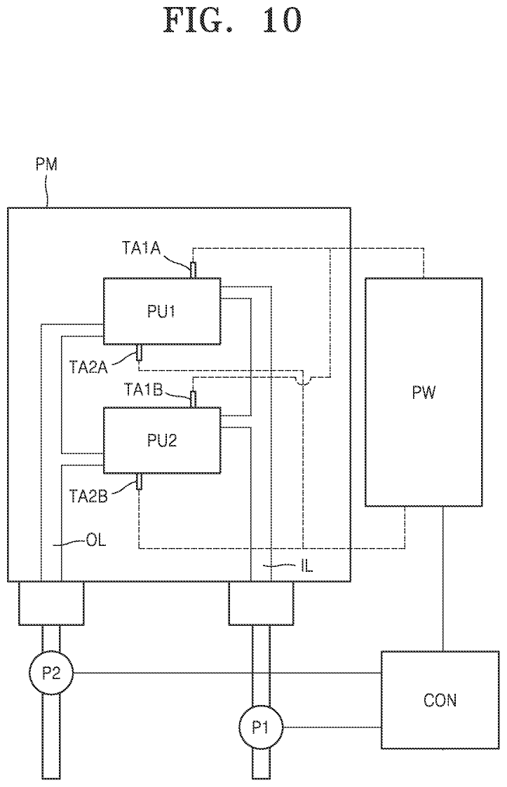

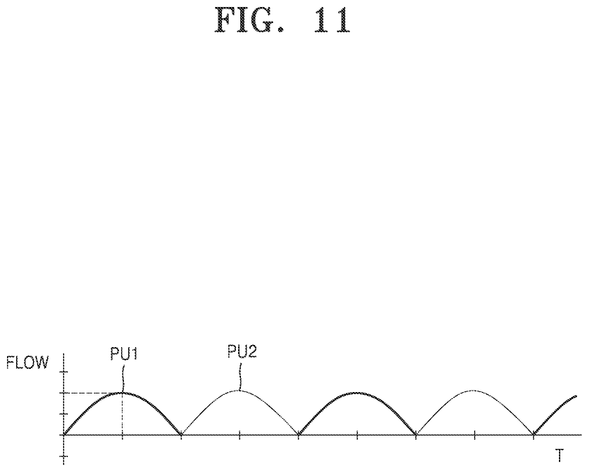

FIG. 10 is a view illustrating a pump module of an exemplary embodiment, which is applied to FIG. 9, and FIG. 11 is a graph illustrating a flow rate of the pump module of FIG. 10. As a pump module PM of FIG. 10, the first pump module PM1 or the second pump module PM2 of FIG. 9 may be applied.

Referring to FIG. 10, the pump module PM may include a plurality of pumps. In the drawing, an exemplary embodiment in which the pump module PM is provided with a first pump PU1 and a second pump PU2 is illustrated, but the disclosure is not limited thereto, and three or more pumps may be provided. However, in the following description, for convenience of description, the exemplary embodiment including two pumps will be mainly described.

The electro-osmosis pump of FIG. 1 or the pump 100 of FIG. 8 described above may be disposed in place of the first pump PU1 and the second pump PU2.

A fluid may be introduced through an inlet line IL, and may be discharged through an outlet line OL. An inlet of the first pump PU1 and an inlet of the second pump PU2 may be connected in parallel to the inlet line IL. An outlet of the first pump PU1 and an outlet of the second pump PU2 may be connected in parallel to the outlet line OL.

A first pressure gauge P1 may be disposed in the inlet line IL, and a second pressure gauge P2 may be disposed on the outlet line OL. Data on pressure sensed by the first pressure gauge P1 and the second pressure gauge P2 may be transmitted to a controller CON.

The controller CON may control driving of a power supply PW. The controller CON may control polarities of voltages applied to the first pump PU1 and the second pump PU2. In addition, the controller CON may adjust a cycle with which the polarities applied to the first pump PU1 and the second pump PU2 change.

The first pump PU1 may be disposed between the inlet line IL and the outlet line OL, and a first operation fluid may be disposed in a first housing.

The first pump PU1 may include the first housing, a first membrane disposed in the first housing, a 1A-th electrode disposed on one side of the first membrane, a 2A-th electrode disposed on the other side of the first membrane, a 1A-th diaphragm disposed to be spaced apart from the 1A-th electrode, a 1A-th check valve configured to move the fluid from the inlet line to the 1A-th diaphragm, and a 2A-th check valve configured to move the fluid from the 1A-th diaphragm to the outlet line.

In the first pump PU1, a 1A-th electrode tab TA1A may be connected to the 1A-th electrode, and a 2A-th electrode tab TA2A may be connected to the 2A-th electrode.

The second pump PU2 may be disposed in parallel to the first pump PU1 between the inlet line IL and the outlet line OL, and a second operation fluid may be disposed in a second housing of the second pump PU2.

The second pump PU2 may include the second housing, a second membrane disposed in the second housing, a 1B-th electrode disposed on one side of the second membrane, a 2B-th electrode disposed on the other side of the second membrane, a 1B-th diaphragm disposed to be spaced apart from the 1B-th electrode, a 1B-th check valve configured to move the fluid from the inlet line to the 1B-th diaphragm, and a 2B-th check valve configured to move the fluid from the 1B-th diaphragm to the outlet line.

In the second pump PU2, a 1B-th electrode tab TA1B may be connected to the 1B-th electrode, and a 2B-th electrode tab TA2B may be connected to the 2B-th electrode.

The power supply PW may supply power to the first pump PU1 and the second pump PU2. The power supply PW may supply voltages of different polarities to the pump module PM.

The power supply PW may provide voltages of different polarities to the 1A-th electrode tab TA1A and the 2A-th electrode tab TA2A of the first pump PU1. The power supply PW may supply a voltage to the 1A-th electrode and the 2A-th electrode of the first pump PU1 by alternating polarities.

The power supply PW may provide voltages of different polarities to the 1B-th electrode tab TA1B and the 2B-th electrode tab TA2B of the second pump PU2. The power supply PW may supply a voltage to the 1B-th electrode and the 2B-th electrode of the second pump PU2 by alternating polarities.

Different polarities may be applied to the 1A-th electrode of the first pump PU1 and the 1B-th electrode of the second pump PU2. The 1A-th electrode and the 2B-th electrode may have one polarity, and the 1B-th electrode and the 2A-th electrode may have the other polarity.

The first operation fluid and the second operation fluid of the first pump PU1 may move in different directions. The fluid may be alternately introduced into the first pump PU1 and the second pump PU2 from the inlet line IL and alternately discharged to the outlet line OL. When the fluid is introduced into the first pump PU1, the pumped fluid may be discharged from the second pump PU2.

Referring to FIG. 11, the pump module PM may continuously pump fluid using the plurality of pumps disposed in parallel. When different polarities are applied to the first pump PU1 and the second pump PU2 of an electro-osmotic method, the first pump PU1 and the second pump PU2 alternately discharge the pumped fluid. Thus, the pumped flow rate may be continuously discharged from the pump module PM within a preset range.

The power supply PW may apply polarities of voltages to the first pump PU1 and the second pump PU2 so that the polarities have different cycles. A discharge cycle of the fluid discharged from the 2A-th check valve of the first pump PU1 and a discharge cycle of the fluid discharged from the 2B-th check valve of the second pump PU2 may be different from each other.

In detail, the first pump PU1 may discharge the fluid from the inlet line IL to the outlet line OL in a first cycle, and the second pump PU2 may discharge the fluid from the inlet line IL to the outlet line OL in a second cycle different from the first cycle.

In an exemplary embodiment, the first cycle and the second cycle may be cycles opposite to each other as shown in FIG. 11.

In another exemplary embodiment, although not shown in the graph, the first cycle and the second cycle may overlap at least in part. Thus, the first pump PU1 and the second pump PU2 may periodically have a time for which the fluid is simultaneously discharged, so that the flow rate discharged from the pump module PM can be precisely controlled.

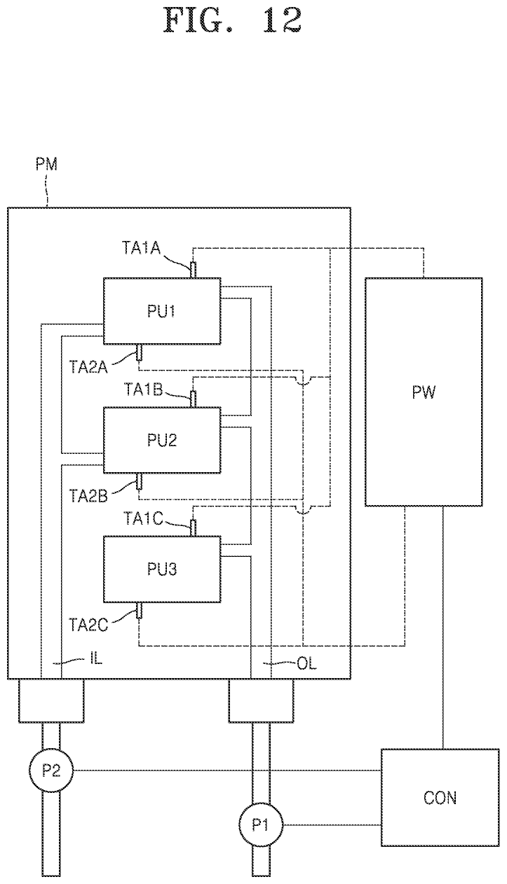

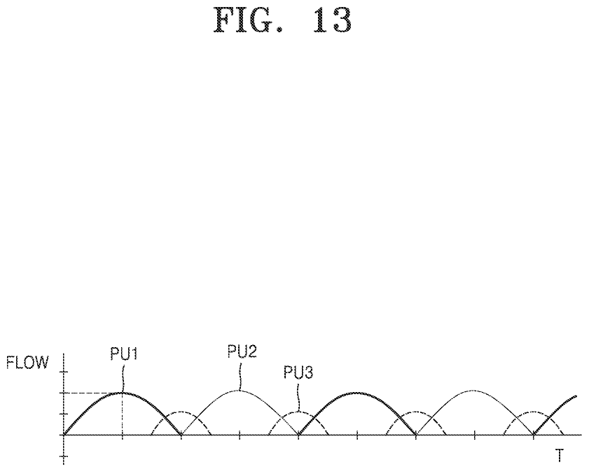

FIG. 12 is a view illustrating a pump module of another exemplary embodiment, which is applied to FIG. 9, and FIG. 13 is a graph illustrating a flow rate of the pump module of FIG. 12.

Referring to FIG. 12, a pump module PM may include three pumps. A first pump PU1 and a second pump PU2 may be substantially the same as those of the pump module described with reference to FIG. 10.

A third pump PU3 may be disposed between an inlet line IL and an outlet line OL. The third pump PU3 may be disposed in parallel to the first pump PU1 and may be disposed in parallel to the second pump PU2.

The power supply PW may supply a voltage to the third pump PU3 by alternating polarities, and may apply the polarity with a cycle that is different from a cycle of the polarity applied to the first pump PU1 and a cycle of the polarity applied to the second pump PU2.

In detail, referring to FIG. 13, a fluid may be alternately discharged from the first pump PU1 and the second pump PU2, and the pumped fluid may be discharged from the third pump PU3 at a time between the discharge times of the first pump PU1 and the second pump PU2. The third pump PU3 discharges the pumped fluid in a section in which the introduction and the discharge of the fluid are switched in the first pump PU1 and a section in which the introduction and the discharge of the fluid are switched in the second pump PU2, so that a variation in the flow rate of the fluid joined in the outlet line OL can be reduced.

In an exemplary embodiment, a flow rate of the fluid discharged from the third pump PU3 may be different from a flow rate discharged from the first pump PU1 or the second pump PU2. For example, the flow rate discharged from the third pump PU3 may be less than the flow rate discharged from the first pump PU1 or the second pump PU2. Since the flow rate discharged from the third pump PU3 is relatively less than that of the first pump PU1 or the second pump PU2, a flow rate of the fluid joined in the outlet line OL can be maintained.

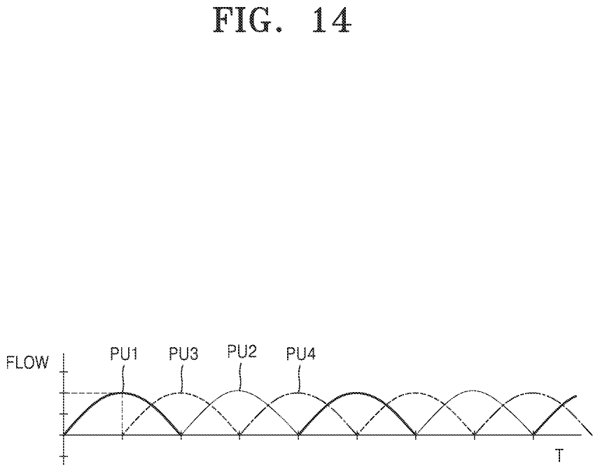

FIG. 14 is a graph illustrating a flow rate of a pump module of another exemplary embodiment, which is applied to FIG. 9.

Referring to FIG. 14, a pump module PM may include four pumps. A first pump PU1, a second pump PU2, a third pump PU3, and a fourth pump PU4 discharge a pumped fluid with different cycles, so that a variation of a flow rate discharged to an outlet line OL can be minimized.

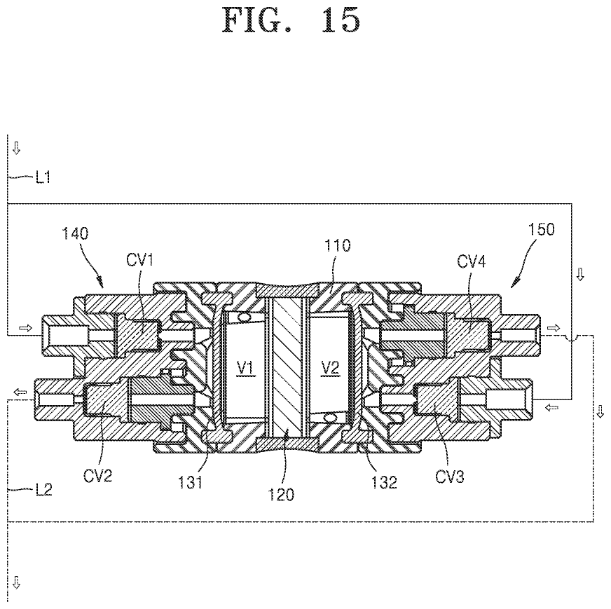

FIG. 15 is a view illustrating a pump module of another exemplary embodiment, which is applied to FIG. 9.

Referring to FIG. 15, the pump 100 described above may be applied to the pump module.

The pump 100 may include a housing 110, a driver 120, a first diaphragm 131, a second diaphragm 132, a first valve assembly 140, and a second valve assembly 150. The first valve assembly 140 may include a first check valve CV1 and a second check valve CV2, and the second valve assembly 150 may include a third check valve CV3 and a fourth check valve CV4.

The pump 100 may be disposed in the first line, and one of blood and dialysate may be alternately introduced into the first check valve CV1 and the third check valve CV3, and the one of the blood and the dialysate may be alternately discharged to the second check valve CV2 and the fourth check valve CV4.

In an exemplary embodiment, the 1A-th line L1 is connected to the first check valve CV1 and the third check valve CV3, so that the blood can be introduced into the pump 100. In addition, the 1B-th line L2 is connected to the second check valve CV2 and the fourth check valve CV4, so that the blood can be discharged from the pump 100.

In an exemplary embodiment, although not shown in the drawing, the 2A-th line C1 is connected to the first check valve CV1 and the third check valve CV3, so that, the dialysate can be introduced into the pump 100. In addition, the 2B-th line C2 is connected to the second check valve CV2 and the fourth check valve CV4, so that the dialysate can be discharged from the pump 100.

Since voltages of different polarities are alternately applied to the pump 100 from the power supply PW, the movement of an operation fluid is switched. In the pump 100, a volume of each of the first space V1 and the second space V2 is changed due to the movement of the operation fluid.

In detail, when the first check valve CV1 is opened, the fourth check valve CV4 is opened, and the second check valve CV2 and the third check valve CV3 are closed. In addition, when the second check valve CV2 is opened, the third check valve CV3 is opened, and the first check valve CV1 and the fourth check valve CV4 are closed.

When a fluid such as blood or dialysate is introduced into the first valve assembly 140, the pumped fluid is discharged from the second valve assembly 150, and when a fluid is introduced into the second valve assembly 150, the pumped fluid is discharged from the first valve assembly 140. Thus, in the pump 100, a fluid can be substantially continuously introduced from the 1A-th line L1 or the 2A-th line C1, and the pumped fluid can be substantially continuously discharged to the 1B-th line L2 or the 2B-th line C2.

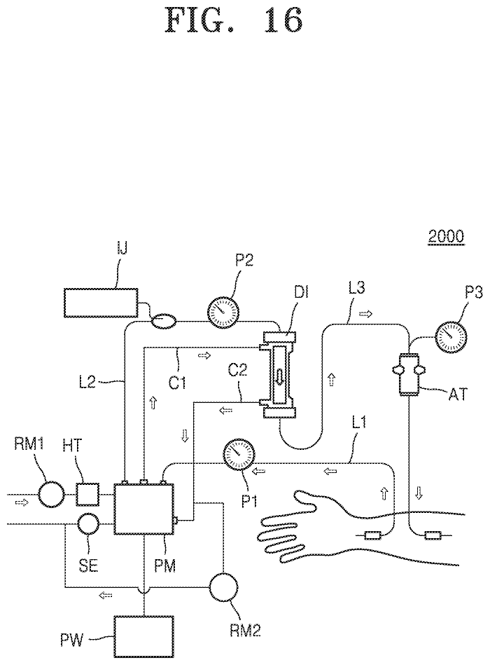

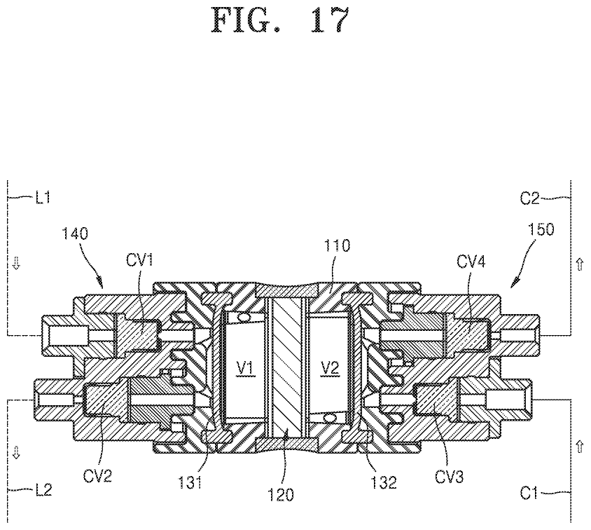

FIG. 16 is a view illustrating a dialysis system according to another exemplary embodiment of the present invention, and FIG. 17 is a view illustrating a pump module of an exemplary embodiment, which is applied to FIG. 16.

Referring to FIGS. 16 and 17, a dialysis system 2000 may include a 1A-th line L1, a 1B-th line L2, and a 1C-th line L3 through which blood moves, and may include a 2A-th line C1 and a 2B-th line C2 through which dialysate moves. In addition, the dialysis system 2000 may include a pump module PM and a dialysis device DI.

In an alternative exemplary embodiment, the dialysis system 2000 may include at least one of a first pressure gauge P1, a second pressure gauge P2, a third pressure gauge P3, a liquid medicine injector IJ, an air trap AT, an air removal pump RM1, a heater HT, a moisture removal pump RM2, and a detector SE.

The dialysis system 2000 is different from the dialysis system 1000 described above in the arrangement of the pump module PM, and thus, this will be mainly described below. The pump module PM may include the pump 100 described above.

The pump 100 may include the first valve assembly 140 disposed in a first line and the second valve assembly 150 disposed in a second line. One of the blood and the dialysate discharged from the second check valve CV2 and the other one of the blood and the dialysate discharged from the fourth check valve CV4 may be alternately discharged.

In detail, the blood may pass through the first valve assembly 140, and the dialysate may pass through the second valve assembly 150. As the driver 120 is driven, the first check valve CV1 of the first valve assembly 140 is opened, the second check valve CV2 is closed, and the blood is introduced into the first valve assembly 140. At the same time, the third check valve CV3 of the second valve assembly 150 is closed, the fourth check valve CV4 is opened, and the pumped dialysate is discharged from the second valve assembly 150. Thereafter, the first check valve CV1 of the first valve assembly 140 is closed, the second check valve CV2 is opened, and the pumped blood is discharged from the first valve assembly 140. At the same time, the third check valve CV3 of the second valve assembly 150 is opened, the fourth check valve CV4 is closed, and the dialysate is introduced into the second valve assembly 150.

In the dialysis system 2000 of the present invention, the pump module PM driven by an electro-osmotic method can pump the blood and the dialysate together. In particular, the blood and the dialysate can be alternately pumped by the driving of the pump module PM, so that the efficiency of the dialysis system 2000 can be increased.

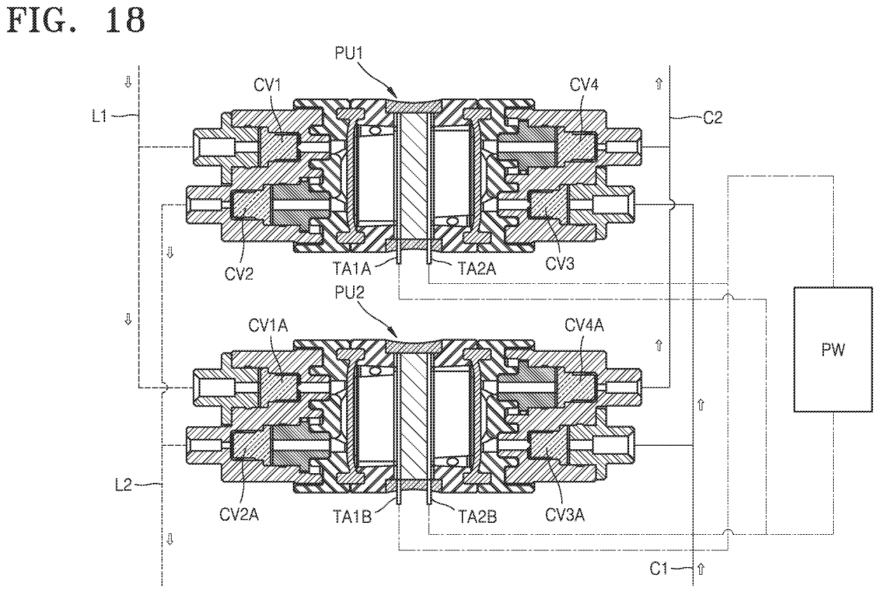

FIG. 18 is a view illustrating a pump module of another exemplary embodiment, which is applied to FIG. 16.

Referring to FIG. 18, a pump module PM may include a plurality of pumps disposed in parallel. In the drawing, two pumps are illustrated as being disposed in parallel, but the disclosure is not limited thereto, and three or more pumps may be disposed.

A first pump PU1 may include a first valve assembly and a second valve assembly. The first valve assembly may include a first check valve CV1 disposed at an inlet end thereof, and a second check valve CV2 disposed at an outlet end thereof. The second valve assembly may include a third check valve CV3 disposed at an inlet end thereof, and a fourth check valve CV4 disposed at an outlet end thereof.

A second pump PU2 may include a 1A-th valve assembly and a 2A-th valve assembly. The 1A-th valve assembly may include a 1A-th check valve CV1A disposed at an inlet end thereof, and a 2A-th check valve CV2A disposed at an outlet end thereof. The 2A-th valve assembly may include a 3A-th check valve CV3A disposed at an inlet end thereof, and a 4A-th check valve CV4A disposed at an outlet end thereof.

The first valve assembly and the 1A-th valve assembly may be disposed in the first line through which blood moves, and the second valve assembly and the 2A-th valve assembly may be disposed in the second line through which dialysate moves.

The first check valve CV1 and the 1A-th check valve CV1A are connected to the 1A-th line L1, so that the blood can be introduced into the first pump PU1 and the second pump PU2. The second check valve CV2 and the 2A-th check valve CV2A are connected to the 1B-th line L2, so that the blood can be discharged from the first pump PU1 and the second pump PU2.

The third check valve CV3 and the 3A-th check valve CV3A are connected to the 2A-th line C1, so that dialysate can be introduced into the first pump PU1 and the second pump PU2. The fourth check valve CV4 and the 4A-th check valve CV4A are connected to the 2B-th line C2, so that the dialysate can be discharged from the first pump PU1 and the second pump PU2.

A first electrode of the first pump PU1 is connected to a 1A-th electrode tab TA1A, and a second electrode of the first pump PU1 is connected to a 2A-th electrode tab TA2A. A 1A-th electrode of the second pump PU2 is connected to a 1B-th electrode tab TA1B, and a 2A-th electrode of the second pump PU2 is connected to a 2B-th electrode tab TA2B.

A power supply PW may supply a voltage to the 1A-th electrode tab TA1A and the 2a-th electrode tab TA2A of the first pump PU1 by alternating polarities, and may supply a voltage to the 1B-th electrode tab TA1B and the 2B-th electrode tab TA2B of the second pump PU2 by alternating polarities.

The power supply PW may supply voltages of different polarities to the first pump PU1 and the second pump PU2. For example, the 1A-th electrode tab TA1A and 1B-th electrode tab TA1B may have different polarities, and the 2A-th electrode tab TA2A and the 2B-th electrode tab TA2B may have different polarities.

In the pump module, a flow direction of a first operation fluid of the first pump PU1 and a flow direction of a second operation fluid of the second pump PU2 are set to be opposite to each other.

The blood can be substantially continuously introduced into and discharged from the pump module along the first line. In addition, the dialysate can be substantially continuously introduced into and discharged from the pump module along the second line.

While the disclosure has been described in connection with what is presently considered to be practical exemplary embodiments, it is to be understood that the disclosure is not limited to the disclosed exemplary embodiments. On the contrary, it is intended to cover various modifications and equivalent arrangements included within the spirit and scope of the appended claims.

What is claimed is:

1. An electro-osmosis pump system comprising:
an inlet line through which a fluid is introduced;
an outlet line through which the fluid is discharged;
a first pump disposed between the inlet line and the outlet line and including a first housing in which a first operation fluid is disposed;
a second pump disposed in parallel to the first pump between the inlet line and the outlet line and including a second housing in which a second operation fluid disposed; and
a power supply configured to supply voltages to the first pump and the second pump,
wherein the first pump includes:
a first membrane disposed in the first housing;
a 1A-th electrode disposed on one side of the first membrane; and
a 2A-th electrode disposed on the other side of the first membrane,
the second pump includes:
a second membrane disposed in the second housing;
a 1B-th electrode disposed on one side of the second membrane; and
a 2B-th electrode disposed on the other side of the second membrane, and
the power supply supplies the voltage to the 1A-th electrode and the 2A-th electrode of the first pump by alternating polarities, and supplies the voltage to the 1B-th electrode and the 2B-th electrode of the second pump by alternating polarities, wherein the polarity applied to the 1A-th electrode of the first pump and the polarity applied to the 1B-th electrode of the second pump are different from each other.

2. The electro-osmosis pump system of claim 1, wherein the first operation fluid of the first pump and the second operation fluid of the second pump move in different directions.

3. The electro-osmosis pump system of claim 1, wherein
the first pump further includes:
a 1A-th diaphragm disposed to be spaced apart from the 1A-th electrode;
a 1A-th check valve configured to move the fluid from the inlet line to the 1A-th diaphragm; and
a 2A-th check valve configured to move the fluid from the 1A-th diaphragm to the outlet line, and
the second pump further includes:
a 1B-th diaphragm disposed to be spaced apart from the 1B-th electrode;
a 1B-th check valve configured to move the fluid from the inlet line to the 1B-th diaphragm; and
a 2B-th check valve configured to move the fluid from the 1B-th diaphragm to the outlet line.

4. The electro-osmosis pump system of claim 3, wherein a discharge cycle of the fluid discharged from the 2A-th check valve of the first pump and a discharge cycle of the fluid discharged from the 2B-th check valve of the second pump are different from each other.

5. The electro-osmosis pump system of claim 3, wherein, in the first pump and the second pump, the fluid is alternately introduced from the inlet line and alternately discharged to the outlet line.

6. The electro-osmosis pump system of claim 1, wherein
the first pump discharges the fluid from the inlet line to the outlet line in a first cycle, and
the second pump discharges the fluid from the inlet line to the outlet line in a second cycle different from the first cycle.

7. The electro-osmosis pump system of claim 1, further comprising a third pump disposed in parallel to the first pump and in parallel to the second pump, between the inlet line and the outlet line,
wherein the power supply supplies a voltage to the third pump by alternating polarities, wherein the polarity is applied in a cycle that is different from a cycle of the polarity applied to the first pump and a cycle of the polarity applied to the second pump.

8. The electro-osmosis pump system of claim 7, wherein a flow rate of the fluid discharged from the third pump is different from a flow rate of the fluid discharged from the first pump or the second pump.

9. A dialysis system comprising:
a first line through which one of blood and dialysate moves;
a second line through which the other one of the blood and the dialysate moves;
a dialysis device through which the first line and the second line pass and in which the blood is dialyzed by the dialysate;
a pump module disposed in at least one of the first line and the second line and configured to move at least one of the dialysis and the blood passing therethrough; and
a power supply configured to alternately supply power to the pump module by alternating polarities.

10. The dialysis system of claim 9, wherein
the pump module includes:
a first pump disposed in the first line and including a first housing in which an operation fluid disposed; and
a second pump disposed in the first line in parallel to the first pump and including a second housing in which an operation fluid disposed,
wherein the first pump includes:
a first membrane disposed in the first housing;
a 1A-th electrode disposed on one side of the first membrane; and
a 2A-th electrode disposed on the other side of the first membrane,
the second pump includes:
a second membrane disposed in the second housing;
a 1B-th electrode disposed on one side of the second membrane; and
a 2B-th electrode disposed on the other side of the second membrane, and
the power supply supplies a voltage to the 1A-th electrode and the 2A-th electrode of the first pump by alternating polarities, and supplies a voltage to the 1B-th electrode and the 2B-th electrode of the second pump by alternating polarities, wherein the polarities are applied to the 1A-th electrode of the first pump and the 1B-th electrode of the second pump with different cycles.

11. The dialysis system of claim 10, wherein the operation fluid of the first pump and the operation fluid of the second pump move in different directions.

12. The dialysis system of claim 10, wherein
the first pump further includes:
a 1A-th diaphragm disposed to be spaced apart from the 1A-th electrode;
a 1A-th check valve configured to move the blood or the dialysate from the first line to the 1A-th diaphragm; and
a 2A-th check valve configured to move the blood or the dialysate from the 1A-th diaphragm to the first line,
the second pump further includes:
a 1B-th diaphragm disposed to be spaced apart from the 1B-th electrode;
a 1B-th check valve configured to move the blood or the dialysate from the first line to the 1B-th diaphragm; and
a 2B-th check valve configured to move the blood or the dialysate from the 1B-th diaphragm to the first line, and
a discharge cycle of the blood or the dialysate discharged from the 2A-th check valve of the first pump or a discharge cycle of the blood or the dialysate discharged from the 2B-th check valve of the second pump are different from each other.

13. The dialysis system of claim 10, wherein
the first pump discharges the blood or the dialysate into the first line in a first cycle, and
the second pump discharges the blood or the dialysate in a second cycle different from the first cycle.

14. The dialysis system of claim 10, further comprising a third pump disposed in parallel to the first pump and in parallel to the second pump,
wherein the power supply supplies a voltage to the third pump by alternating polarities, wherein the polarity is applied in a cycle that is different from a cycle of the polarity applied to the first pump and a cycle of the polarity applied to the second pump.

15. The dialysis system of claim 14, wherein a flow rate of the blood or the dialysate discharged from the third pump is different from a flow rate of the blood or the dialysate discharged from the first pump or the second pump.

16. The dialysis system of claim 9, wherein
the pump module includes at least one pump,
wherein the pump includes:
a housing;
a driver including a first membrane disposed in the housing, a first electrode disposed on one side of the first membrane, and a second electrode disposed on the other side of the first membrane;
a diaphragm assembly including a first diaphragm disposed on one side of the driver and a second diaphragm disposed on the other side of the driver;
a first valve assembly mounted to face the first diaphragm and including a first check valve disposed at an inlet end and a second check valve disposed at an outlet end; and
a second valve assembly mounted to face the second diaphragm and including a third check valve disposed at an inlet end and a fourth check valve disposed at an outlet end.

17. The dialysis system of claim 16, wherein
the pump is disposed in the first line, and
one of the blood and the dialysate is alternately introduced into the first check valve and the third check valve, and is alternately discharged from the second check valve and the fourth check valve.

18. The dialysis system of claim 16, wherein
in the pump,
the first valve assembly is disposed in the first line,
the second valve assembly is disposed in the second line,
one of the blood and the dialysate discharged from the second check valve, and
the other one of the blood and the dialysate discharged from the fourth check valve are alternately discharged.
